(19) United States
(12) Patent Application Publication
(10) Pub. No.: US 2024/0011068 A1
(43) Pub. Date: Jan. 11, 2024

(54) SILICON MEMBRANE WITH INFRARED TRANSMITTANCE AND PROCESS FOR MANUFACTURING

(71) Applicant: COMMISSARIAT A L'ENERGIE ATOMIQUE ET AUX ENERGIES ALTERNATIVES, Paris (FR)

(72) Inventors: Frederic-Xavier GAILLARD, Grenoble Cedex 09 (FR); Yoko OTSUKA, Grenoble Cedex 09 (FR)

(73) Assignee: COMMISSARIAT A L'ENERGIE ATOMIQUE ET AUX ENERGIES ALTERNATIVES, Paris (FR)

(21) Appl. No.: 18/348,077

(22) Filed: Jul. 6, 2023

(30) Foreign Application Priority Data
Jul. 8, 2022 (FR) 22 07005

(51) Int. Cl. C12Q 1/02; C25F 3/12; C12M 1/12

(52) U.S. Cl. CPC C12Q 1/02; C25F 3/12; C12M 25/02

(57) ABSTRACT

Fluid transfer membrane (84) including a porous wall (82) of n-doped silicon including pores (54) extending entirely across its thickness, each pore having a diameter of less than or equal to 400 nm and an aspect ratio of greater than or equal to 20.

The present invention relates to the production of a highly elongated porous structure for the manufacture of a porous membrane for the transfer of a fluid, and which is capable of transmitting infrared radiation, notably for the purpose of measuring a physical magnitude in the infrared which is characteristic of an object or a living organism.

In the field of life sciences and environmental sciences, it is known practice to characterize modes of vibration or to measure absorption spectra in the infrared in order, for example, to discriminate healthy tissue from pathological tissue, to locate tumours on anatomo-pathological samples, to identify pathogenic elements in a tissue, or to assay undesirable chemical compounds such as pesticides or endocrine disruptors.

For example, it is known practice from FR 3 108 983 A1 to characterize colonies of microorganisms grown on an aluminium oxide membrane, using infrared radiation and transmission through the membrane. Such a membrane is porous and transfers the nutrient medium to the microorganisms in the pores that pass through its thickness. However, aluminium oxide is totally opaque to infrared wavelengths greater than 10 μm, which limits the amount of information that may be acquired to characterize the colony.

The invention is thus directed towards proposing a novel membrane for transferring a fluid, for example water, an alcohol or a nutrient medium for culturing microorganisms, and which has infrared transmittance over a wider range of wavelengths than an alumina membrane, and also a process for manufacturing such a membrane.

The invention relates to a fluid transfer membrane including a porous wall of n-doped silicon including pores extending entirely across its thickness, each pore having a diameter of less than or equal to 400 nm and an aspect ratio of greater than or equal to 20.

The membrane thus transmits radiation over a wider range of wavelengths than an alumina membrane. Preferably, the membrane has a transmittance of greater than 5% to any radiation with a wavelength of between 5 μm and 25 μm.

Preferably, the membrane has a transmittance of greater than or equal to 10%, or even greater than or equal to 20%, better still greater than or equal to 30%, to any radiation with a wavelength of between 5 μm and 25 μm.

The membrane may have a transmittance of greater than or equal to 20% to any radiation with a wavelength of between 10 μm and 25 μm.

Preferably, each pore has a diameter of less than or equal to 350 nm and/or greater than or equal to 50 nm.

Preferably, the pores have an aspect ratio of greater than or equal to 50, better still greater than or equal to 100.

Preferably, the pores extend transversely between the faces of the wall onto which they open, i.e. substantially perpendicular to said faces.

The shape of the pores is not limiting. The pores may vary in shape, notably tubular, for example rotationally cylindrical.

The pores may have a diameter which varies along their axis of extension between the apertures through which they open, preferably by 100% at most in absolute value. In particular, the pores may have lateral faces which undulate along their axis of extension between the apertures through which they open.

Preferably, the length of each pore is greater than or equal to 10 μm, preferably greater than or equal to 30 μm, better still greater than or equal to 50 μm.

The pore apertures may be of varied shape. In particular, they may be convex, notably polygonal, for example square, elliptical, circular or cross-shaped.

Preferably, the pore surface density is between 0.1 μm-2 and 5.0 μm⁻². The porous wall may have a thickness of greater than or equal to 10 μm, preferably greater than or equal to 30 μm, better still greater than or equal to 50 μm.

The porous wall is n-doped, for example with phosphorus.

Preferably, the membrane has the general shape of a plate, preferably a flat plate extending between two large faces.

The angle between the opposite large faces of the membrane is less than or equal to 5°. In particular, the opposite large faces of the membrane may be parallel.

Preferably, to improve the transmittance of the membrane, at least one, preferably each of the faces of the membrane presents a surface roughness of less than 20 nm. The surface roughness may be measured by atomic force microscopy.

Preferably, at least one portion, preferably the entirety of the area of at least one, preferably of each of the opposite large faces of the membrane, is free of any covering coating it. Such a coating reduces the transmittance of the membrane.

The membrane may be between 20 μm and 90 mm long. For example, it is suitable for the characteristics of an infrared analysis device, for example for the size of the beam, on which it is mounted.

The membrane may consist of the porous wall. In a preferred variant, the membrane also includes a support superimposed on the porous wall and which includes at least one recess passing through the support entirely across its thickness, at least one pore opening into the recess. The support thus imparts rigidity and mechanical strength to the membrane, making it easier to grip and handle. Preferably, the recess length, measured in a median plane of the membrane, is greater than 1 mm. It may be greater than 3 mm, even greater than 5 mm. Thus, the recess is long enough for its interaction with a radiation induces little or no reduction of the membrane transmittance.

Preferably, the support and the porous wall extend in parallel planes. The thickness of the support may be between 100 μm and 3 mm, notably less than or equal to 1 mm.

The support may include, or even consist of, silicon. It may be made of the same material as the porous wall.

The support may be in contact with the porous wall.

The support may be bonded to the porous wall. It may be bonded via a direct hydrophobic bonding technique, notably by silicon-to-silicon bonding. As a variant, it is bonded via a hydrophilic surface bonding technique, for example using silicon oxide. The surfaces may be deposited, prepared by nitrogen or oxygen plasma activation or by a mechanochemical polishing process. Bonding may involve other types of material such as organic materials of polymeric origin or metallic materials.

Preferably, the support is dense, i.e. it has a total porosity of less than 0.1%, the total porosity of the support being equal to the ratio of the total volume of pores in the support divided by the volume of the support.

The support and the wall may form a monolithic assembly.

The invention also relates to a process for manufacturing the membrane according to the invention, the process involving:
the provision of an n-doped silicon substrate which has an electrical resistivity of between 1 Ω·cm and 10 Ω·cm, and of an electrochemical cell including an electric generator, a cathode and an anode electrically powered by the electric generator, the electrochemical cell containing an aqueous electrolyte including hydrofluoric acid in a mass concentration of greater than or equal to 10%,
   the substrate being arranged between the anode and the cathode,
   at least one of the faces of the silicon substrate being in contact with the electrolyte, the cathode and the face of the silicon substrate opposite the cathode, known as the “front face”, being in contact with the electrolyte, the front face of the silicon substrate being distant from the cathode,
electrochemical etching of the substrate by applying with the electric generator a constant electric voltage of greater than or equal to 10 V between the anode and the cathode, so as to form blind pores in the substrate, each of which has a diameter of less than or equal to 400 nm and which extend from one face of the substrate to a depth of greater than or equal to 10 μm, the rear face of the substrate being illuminated during the electrochemical etching with light radiation so as to generate charge carriers in the substrate, and
ablation of part of the substrate so that said pores are open and pass through at least a portion of the substrate entirely across its thickness.

The process according to the invention thus allows the manufacture of a membrane in which the pores have a diameter of less than or equal to 400 nm, preferably between 50 nm and 350 nm. Such pores, which are more than ten times smaller than the wavelengths of infrared radiation between 5 μm and 25 μm, do not substantially scatter and/or diffract this radiation, which may thus be readily transmitted through the membrane. In addition, implementation of the process according to the invention allows rapid production of the membrane with a pore etch rate preferably greater than 4 μm·min⁻¹.

Preferably, the substrate has an electrical resistivity of between 3 Ω·cm and 6 Ω·cm.

The aqueous electrolyte is liquid. Preferably, it has a hydrofluoric acid mass concentration of greater than or equal to 15%, preferably greater than or equal to 25%, more preferably greater than or equal to 30%, better still greater than or equal to 35%. The mass concentration of hydrofluoric acid in the aqueous electrolyte may be less than or equal to 50%.

The aqueous electrolyte may be obtained by diluting in an aqueous solvent, notably water, at least one compound chosen from hydrogen fluoride, ammonium fluoride and mixtures thereof.

The aqueous electrolyte may also contain an additive, such as isopropanol and/or acetic acid.

The anode and/or cathode may be metallic, for example made of platinum. As a variant, the anode and/or cathode may be made of silicon.

Prior to electrochemical etching, the process may involve illuminating the substrate with light radiation so as to generate charge carriers in the substrate.

Prior to electrochemical etching, the substrate may have a thickness of between 100 μm and 3 mm, for example between 500 μm and 1.5 mm.

The substrate may have an orientation (100) or (111) parallel to a normal to one of its faces. It preferably has the shape of a flat plate.

Preferably, the cathode has a substantially flat face which is entirely superimposed on the front face of the substrate. Preferably, the face of the cathode facing the front face of the substrate and said front face are parallel.

According to a first variant, the anode is at a distance from the “rear face” of the substrate, which is opposite the front face of the substrate.

Preferably, the anode is not superimposed on the “rear face” of the substrate.

The electrolytic cell may define first and second leaktight chambers which are closed off by the substrate, the first chamber containing the electrolyte, the second chamber including the electrolyte or another aqueous electrolyte. The front face and rear face of the substrate respectively define a wall of the first chamber and a wall of the second chamber. As the first and second chambers are leaktight, the liquids contained in these chambers do not mix.

Moreover, so as to illuminate the rear face of the substrate, the electrochemical cell may include a window which is transparent to the illuminating radiation and which delimits the second chamber.

The substrate may be completely immersed in the electrolyte. The rear face of the substrate, opposite the front face, is thus placed in contact with the electrolyte. Each of the first and second chambers contains the electrolyte. Alternatively, the second chamber may include another aqueous electrolyte different from the electrolyte contained in the first chamber. This other electrolyte is, for example, an aqueous solution of potassium sulfate K₂SO₄.

According to a second variant, the anode is placed in contact with the rear face of the substrate and partially covers the rear face of the substrate.

Illumination of the substrate is preferably performed with light radiation having an energy of greater than 1.1 eV. For example, it is produced by one or more halogen lamps with a luminous power of greater than or equal to 500 lumens.

Electrochemical etching is performed at a constant electrical voltage, i.e. in potentiostatic mode. Preferably, it is performed at a constant electrical voltage of between 25 V and 100 V.

Preferably, electrochemical etching is performed until the pores each extend from one face of the substrate to a depth greater than or equal to 30 μm, preferably greater than or equal to 50 μm, or even greater than or equal to 75 μm.

Preferably, the part of the substrate which is ablated extends from the face of the substrate opposite the face on which the blind pores open to the bottom of the blind pores.

Prior to the ablation step, the process may include mechanochemical polishing of the face of the substrate onto which the blind pores open, so as to abrade a nanoporous silicon surface layer having a thickness of between 50 nm and 1 μm, formed on said face during electrochemical etching.

Moreover, ablation may be performed by mechanical polishing, wet chemical etching and/or dry deep etching.

Preferably, the ablation is selective so as to extract only part of the portion of the substrate superimposed on the blind pores, preferably to form at least one recess via which at least one, preferably several, of the pores emerge.

Preferably, the selective ablation involves deep etching of the substrate. Deep etching involves applying a protective mask, preferably by photolithography of a photoresist resin, to the face of the substrate opposite the face from which the blind pores open. The protective mask includes at least one window passing entirely across its thickness. The deep etching may be chemical and involve placing the face of the substrate covered with the protective mask in contact with a chemical ablation agent, for example an aqueous composition based on hydrofluoric acid, so as to extract material from an area of the substrate superimposed on the window(s). As a variant, the deep etching may be physical and involve ablation of the material in an area of the substrate superimposed on the window(s) by bombardment of the face of the substrate covered with the protective mask by means of a plasma. The plasma may include an ionized reactive gas, notably a fluoro gas, for example chosen from SF₆, CF₄ and mixtures thereof.

Preferably, prior to the ablation step, the process may include the deposition of a protective layer, preferably formed from at least one oxide, notably silicon oxide, for example deposited by chemical vapour deposition (CVD) using tetraethyl orthosilicate (TEOS) or SiH₄ (silane), on the face of the substrate onto which the support opens. The process may also include removal of the protective layer after the ablation step.

The process may include a subsequent treatment of the membrane, for example a functionalization treatment, to render the membrane hydrophilic or hydrophobic, so as to facilitate the diffusion of specific chemical species.

The invention also relates to a membrane obtained via the process according to the invention.

Finally, the invention relates to a method for characterizing a colony of microorganisms, involving:
culturing the microorganisms in a nutrient medium, the microorganisms being arranged on one face of a membrane according to the invention and/or obtained via the process according to the invention, the membrane being in contact with the nutrient medium in such a way that the nutrient medium is transferred via the pores to the microorganisms,
illuminating the microorganisms in transmission through the membrane with light radiation having at least one wavelength of between 5 μm and 25 μm and acquiring the light radiation which has interacted with the microorganisms.

The “transmittance” T of a body of incident radiation of wavelength λ is equal to the ratio of the intensity I of the transmitted radiation of wavelength λ to the intensity I₀ of the incident radiation, i.e.:

$$T = \frac{I}{{I}_{0}} \qquad [ Math 1 ]$$

The transmittance T may be measured by infrared spectrometry.

The transmittance may be related to several structural features of the membrane, for instance the porous structure, the rugosity of the membrane surfaces, the width and/or the length of raised or recessed reliefs formed on the membrane surface.

The “aspect ratio” of a pore is equal to the ratio of the length of the pore to the diameter of the pore.

The “diameter” of a pore is equal to the average of the diameters of the pore apertures on the two respective opposite faces of the wall onto which the pore opens, or if the pore is blind, the diameter of the pore is equal to the diameter of the aperture through which it opens. The diameter of the aperture of a pore is equal to the diameter of the smallest circle circumscribing the aperture of said pore.

The “length” of a pore is the distance between the apertures through which the pore opens onto opposite faces of the wall, or if the pore is blind, the distance between the aperture through which the pore opens and the bottom of the pore.

The pore “surface density” is determined on a face of the wall or substrate, as the case may be. It is equal to the ratio of the total area occupied by the apertures of the pores on a zone of the face of the wall or of the substrate, respectively, to the area of said zone. Preferably, the area of said zone over which the pore surface density is determined is between 2 μm² and 500 μm².

The “resistivity” of the substrate is measured by the four-point method.

The hydrofluoric acid “mass concentration” of the aqueous electrolyte corresponds to the ratio of the mass of hydrofluoric acid in the aqueous electrolyte to the mass of the aqueous electrolyte.

The “etch rate” of the pores is equal to the ratio of the length of a pore to the duration of the etching step.

The “anode” is an electrode where an oxidation reaction occurs in the electrochemical sense, i.e. electron transfer from the electrolyte to the electrode, or hole transfer from the electrode to the electrolyte.

The “cathode” is an electrode where a reduction reaction occurs in the electrochemical sense, i.e. electron transfer from the electrode to the electrolyte.

The invention may be understood more clearly on reading the detailed description and the drawing which follows, in which:

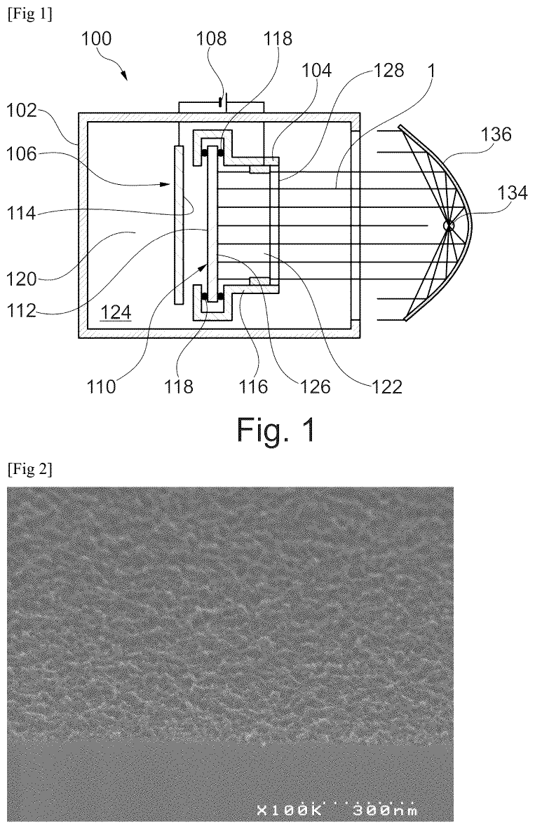

FIG. 1 represents an example of an electrochemical cell for performing the process according to the invention,

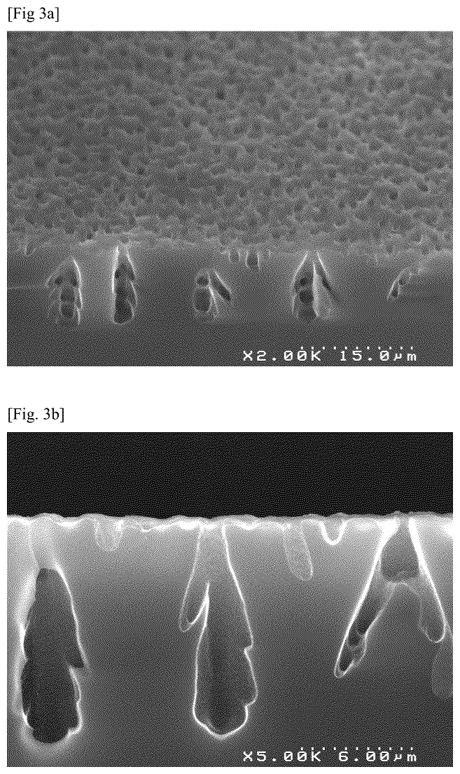

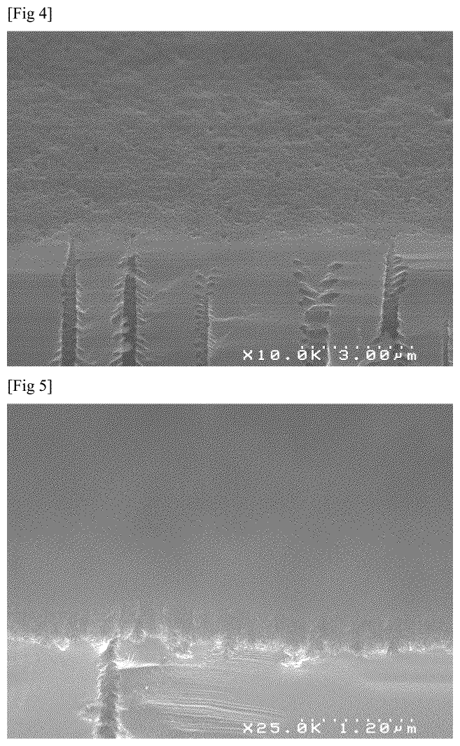

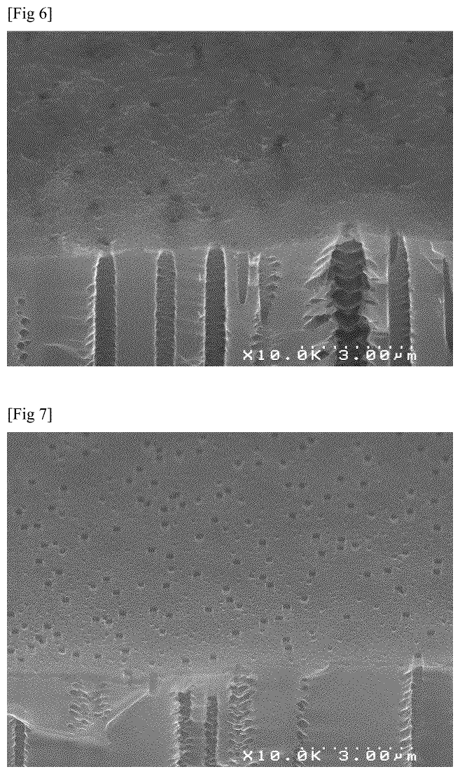

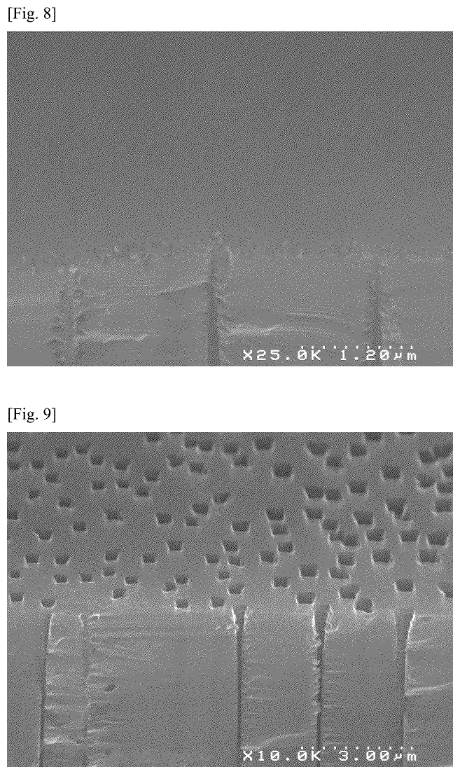

FIG. 2, FIG. 3a, FIG. 3b, FIG. 4, FIG. 5, FIG. 6, FIG. 7, FIG. 8 and FIG. 9 are photographs acquired by scanning electron microscopy of the etched substrates of Examples 1 to 4 and 6 to 9, respectively,

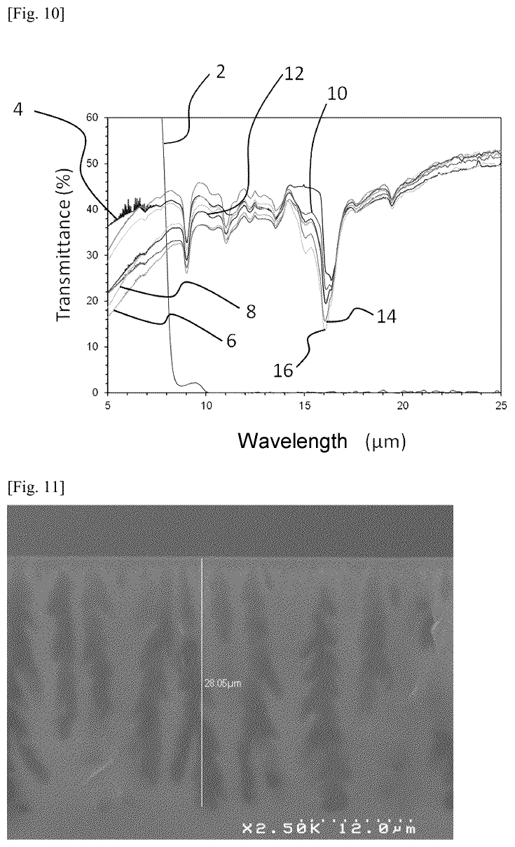

FIG. 10 represents the evolution of the transmittance of different substrates as a function of the wavelength,

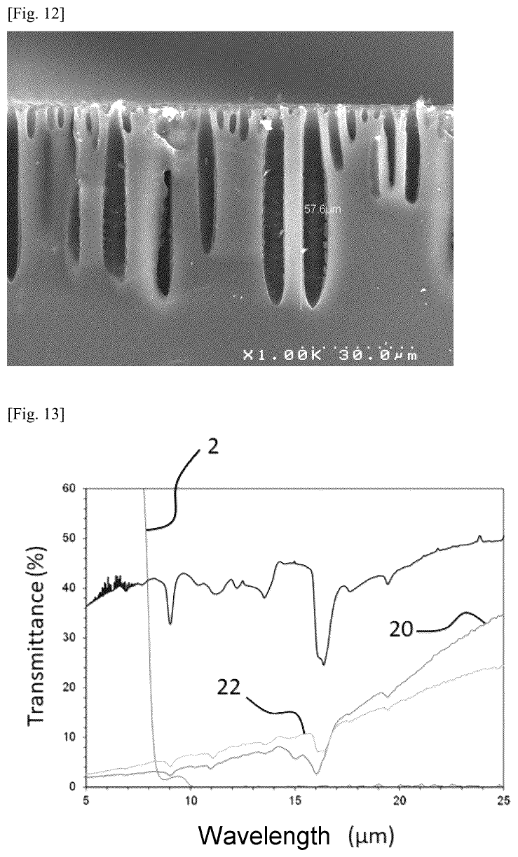

FIG. 11 and FIG. 12 are scanning electron microscopy photographs of the etched substrates of Examples 10 and 11, respectively,

FIG. 13 represents the evolution of the transmittance of the substrates of Examples 10 and 11 as a function of the wavelength,

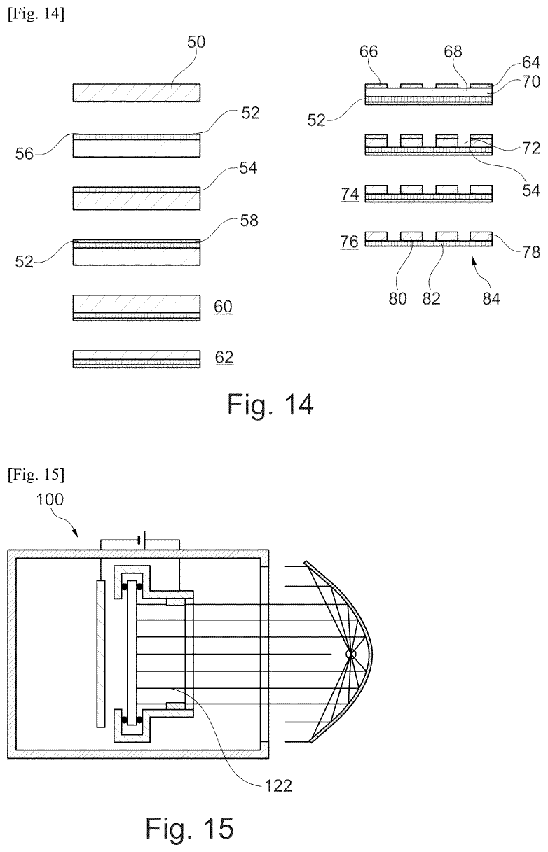

FIG. 14 schematically represents the steps for producing a membrane according to an example of implementation of the process, and

FIG. 15 illustrates another example of an electrochemical cell for performing the process according to the invention.

There is an abundance of scientific literature relating to the production of silicon membranes for multiple applications, for example as summarized in the review article “Porous silicon membranes and their applications: recent advances”, R. Vercauteren et al., Sensors and Actuators, A318 (2021), 112486. For example, the articles
“Thick macroporous membranes made of p-type silicon”, J. Zheng et al., Phys. Status, Solidi. A, vol. 202, num. 8, pages 1402-1406 (2005),
“Integration of low-loss inductors on thin porous silicon membranes”, B. Bardet et al., Microelectron. Eng., vol. 194, pages 96-99 (2018),
“Highly insulating, fully porous silicon substrates for high temperature micro-hotplates”, F. Lucklum et al., Sensor. Actuat. A Phys., vol. 213, pages 35-42 (2014),
“All-in-One Nanowire-Decorated Multifunctional Membrane for RapidCell Lysis and Direct DNA Isolation”, H. So et al., ACS Appl. Mater. Interfaces, vol. 6, pages 20693-20699 (2014),
“Electrochemically etched nanoporous silicon membrane for separation of biological molecules in mixture”, N. Burham et al., J. Micromech. MicroEng., vol. 27, page 075021 (2017), and
“Effects of nano/microstructures on performance of Si-based microfuel cells”, Appl. Therm. Eng., Vol. 72, num. 2, pages 298-303 (2014)
   describe various processes for manufacturing silicon membranes.

The process according to the invention involves electrochemical etching of a substrate in an aqueous electrolyte based on hydrofluoric acid. In general, the electrochemical etching of silicon is a technique that has been known and documented for a long time, for instance in the book “Electrochemistry of Silicon: Instrumentation, Science, Materials and Applications”, Dr. Volker Lehmann, doi: 10.1002/3527600272, 2002 Wiley-VCH Verlag GmbH.

The conditions for performing the process according to the invention are, however, unconventional. To the best of the inventors' knowledge, prior to the invention, work on the electrochemical etching of silicon had mainly been directed towards the search for pores with a regular profile and walls. In this context, studies for these substrate characteristics were mainly focused on pores with a diameter greater than 1 μm and up to about 10 μm and with tubular shapes that were as regular as possible and free from defects, without any interest being paid to the infrared transmission properties. To this end, etchings of silicon substrates were performed under conditions different from those of the invention, for example in galvanostatic mode or in potentiostatic mode but at a low electrical voltage of less than 10 V, and in an aqueous electrolyte where the mass concentration of hydrofluoric acid was low, i.e. less than 10%.

### EXAMPLES

Comparative Examples 1 and 2 and Examples 3 to 9 according to the invention were performed using the electrochemical cell illustrated in FIG. 1.

The electrochemical cell 100 includes an enclosure 102, a silicon anode 104 and cathode 106 being arranged in the enclosure and electrically connected to an electric generator 108. A silicon substrate 110 was housed between the anode and cathode. The front face 112 of the substrate faces a face 114 of the cathode.

The chamber also includes a case 116 with solid walls, the silicon substrate closing off the case. Seals 118 are arranged on either side of the ends of the substrate. Thus, first 120 and second 122 chambers are defined, which are leaktight with respect to each other. An aqueous electrolyte 124 including hydrofluoric acid, the mass concentration of which was modified for the different tests, was introduced into each of the two chambers. Thus, the front 110 and rear 126 faces of the substrate were in contact with the electrolyte, and thus acted as electrodes to ensure electrical continuity during the redox reactions.

Moreover, the part 128 of the wall of the case superimposed on the rear face 126 is transparent to light, and illumination I was generated with halogen lamps 134 and distributed over the entire rear face of the substrate by means of a reflecting mirror 136 of hemispherical shape, so as to generate charge carriers in the substrate.

The substrate was a silicon wafer with a thickness of 750 μm, which was made of phosphorus-doped silicon, and was thus n-type, and had an electrical resistivity of between 3 Ω·cm and 6 Ω·cm.

During the electrochemical etching process, the wafer was illuminated with light radiation of power of greater than 20 000 lumens using halogen lamps.

Electrochemical etching of the front face of the substrate was performed in a potentiostatic regime, with a voltage kept constant between the anode and cathode of the cell entirely across etching.

The duration of the electrochemical etching was kept constant at 6 minutes for all the tests, except for tests 2 and 6 for which the duration was 15 minutes.

Table 1 below summarizes the experimental conditions and the results obtained. The tests marked with an asterisk are comparative. “N.A.” means “not applicable”

TABLE 1

| | | Mass | | | | |
|---|---|---|---|---|---|---|
| | | concen- | | | | |
| | | tration of | Pore | | Pore | |
| | | hydro- | surface | Pore | etch | Pore |
| | Voltage | fluoric | density | diameter | rate | length |
| Test | (V) | acid (%) | (μm⁻²) | (nm) | (μm/min) | (μm) |
| 1⁽*⁾ | 5 | 15 | N.A | N.A. | N.A. | N.A. |
| 2⁽*⁾ | 15 | 5 | 0.05 | 1600 | 0.8 | ~15 μm |
| 3 | 15 | 15 | 0.20 | 230 | 5.3 | ~32 μm |
| 4 | 15 | 35 | 0.38 | 320 | 10.1 | ~50 μm |
| 5 | 25 | 15 | 0.45 | 230 | 5.8 | ~35 μm |
| 6⁽*⁾ | 35 | 5 | 0.35 | 420 | 1.1 | ~15 μm |
| 7 | 35 | 15 | 0.65 | 220 | 6.5 | ~40 μm |
| 8 | 35 | 35 | 0.50 | 150 | 9.7 | ~60 μm |
| 9 | 45 | 35 | 0.85 | 300 | 11.2 | ~65 μm |

In the case of Example 1, outside the invention, the application of a low voltage of 5 V, as conventionally applied prior to the invention, with a hydrofluoric acid mass concentration of 15%, does not allow etching of the substrate to be performed. As can be seen in FIG. 2, the observed face of the substrate has been chemically etched. The result is a rough surface covered with silicon aggregates homogeneously distributed over the surface, but no pores have been formed.

In the case of Example 2, outside the invention, the application of a voltage of V, which was unconventional prior to the invention, with a hydrofluoric acid mass concentration of 5%, results in the slow formation of pores with a diameter of about 1600 nm, which can be seen in FIG. 3a and in the enlargement of FIG. 3b. Increasing the voltage to 35 V, as in Example 6 outside the invention, does not allow a pore size of 400 nm at most to be achieved as according to the invention, as can be seen in FIG. 6. In addition, in these comparative examples, the etch rate is too slow, less than 2 μm/min.

As may be seen from FIGS. 4, 5 and 7 to 9, corresponding to Examples 3, 4 and 7 to 9, performing the process according to the invention allows a substrate to be produced in which the pores have a diameter of between 50 nm and 400 nm.

In the examples illustrated above, at the end of electrochemical etching, the pores obtained were blind and led to a single aperture on one face of the substrate.

Transmittance measurements were performed on the substrates etched in this manner.

The transmittance of the etched substrates, which are the subjects of Examples 3 to 5 and 7 to 9, was measured over the spectrum of infrared wavelengths between 5 μm and 25 μm and is represented in FIG. 10.

Curve 2 represents the change in transmittance of a porous alumina layer including cylindrical through pores with a diameter of between 20 nm and 200 nm, a thickness of 60 μm and a surface density ranging between 10 and 2000 μm⁻².

As shown in FIG. 10, this alumina layer has high transmittance up to a wavelength of 10 μm, but is totally opaque to radiation of longer wavelengths.

The evolution of transmittance for a dense n-type substrate with a resistivity of 3-6 Ω·cm is illustrated by curve 4. This substrate has a transmittance of greater than 20% over the entire wavelength range between 5 μm and 25 μm. However, as it has no pores, it cannot form a membrane for fluid transfer.

Curves 6 to 16 illustrate the evolution of transmittance for Examples 3, 4, 5, 7, 8 and 9 according to the invention, respectively. As can be seen in FIG. 10, the etched substrates in these examples thus have a transmittance close to that of the dense substrate. This confirms that pores with a diameter and aspect ratio according to the invention interact only slightly with infrared radiation, and allow the good infrared transmission properties in the wavelength range from 5 μm to 25 μm of the n-doped silicon substrate to be maintained.

For comparative purposes, n-type substrates identical to those of Examples 1 to 9 were subjected to an electrochemical etching step in the same electrochemical cell, but under galvanostatic conditions, i.e. with a constant current density imposed. Thus, Example 10 was produced, using the same experimental device, by immersing the anode in the hydrofluoric acid solution at a concentration of 35% and then performing the electrochemical etching at a constant current density of 14 mA·cm⁻². Example 11 was performed under conditions identical to those of Example 10, except that the hydrofluoric acid concentration was 15% and the constant current density imposed was 20 mA·cm⁻².

As can be seen in FIGS. 11 and 12, blind pores were formed, but all had a pore diameter greater than 2 μm. These large pores interact with the infrared radiation and reduce the transmittance of the n-type substrate. This can be seen in FIG. 13. Curves 20 and 22 represent the transmittance evolution of Examples 10 and 11, respectively. The transmittance of these etched substrates, outside the invention, is less than 5% for radiation with wavelengths between 5 μm and 8 μm. It is less than 10% for radiation with a wavelength of less than 17 μm for Example 10 and less than 14 μm for Example 11.

Moreover, a membrane was formed for a porous wall obtained under the conditions of Example 3, as illustrated in FIG. 14.

The face 52 of the substrate 50 onto which the blind pores 54 opened was polished so as to remove a layer 56 of nanoporous silicon formed on the surface by electrochemical etching. Next, a protective layer 58 made of silicon oxide (of the TEOS or SiH₄ type deposited by CVD) was deposited on the polished face 52 to prevent the pore apertures from being damaged by the subsequent ablation. The substrate was turned over (step 60) and then mechanically abraded (step 62) to reduce the thickness of the substrate. A layer of photoresist resin 64 was then deposited on the face 66 of the substrate opposite the face 52, and was ablated locally under the effect of ultraviolet light radiation via conventional photolithography processes. Zones 68 of the portion 70 of the substrate superimposed on the blind pores were ablated by deep plasma etching, so as to form recesses 72 in the substrate, delimited by a mask, until the pores 54 opened out into the recess. The photoresist resin and the oxide layer were then removed (steps 74 and 76).

Thus, the non-ablated zones 78 of the substrate form a support 80 provided with recesses, on which a wall 82 is superimposed, provided with blind pores opening onto the non-ablated zones and pores passing entirely across the thickness of the wall and opening via one of their apertures into one of the recesses. A membrane 84 is thus obtained.

The invention is not limited to the examples and optional embodiments and implementation methods presented for illustrative and non-limiting purposes.

Notably, the process may be performed using the electrochemical cell illustrated in FIG. 15. The cell 100 illustrated in FIG. 15 differs from that illustrated in FIG. 1 in that the second chamber 122 contains another electrolyte, for example based on potassium sulfate, different from that contained in the first chamber.

1. Fluid transfer membrane (84) including a porous n-doped silicon wall (82) including pores (54) extending entirely across its thickness, each pore having a diameter of less than or equal to 400 nm and an aspect ratio of greater than or equal to 20, the membrane having a transmittance of greater than or equal to 5% to any radiation whose wavelength is between 5 μm and 25 μm.

2. Membrane according to claim 1, having a transmittance of greater than or equal to 10%, or even greater than or equal to 20%, better still greater than or equal to 30%, to any radiation with a wavelength of between 5 μm and 25 μm.

3. Membrane according to claim 1, each pore having a diameter of less than or equal to 350 nm and/or greater than or equal to 50 nm.

4. Membrane according to claim 1, the pores having an aspect ratio of greater than or equal to 50.

5. Membrane according to claim 1, the pore surface density being between 0.1 μm⁻² and 5.0 μm⁻².

6. Membrane according to claim 1, further including a support (80) superimposed on the porous wall and which includes at least one recess (72) passing through the support entirely across its thickness, at least one pore (54) opening into the recess (72).

7. Membrane according to claim 6, the recess length, measured in a median plane of the membrane, being greater than 1 mm.

8. Membrane according to claim 1, the angle between the opposite large faces of the membrane being less than or equal to 5°.

9. Membrane according to claim 1, at least one of the faces of the membrane presenting a surface roughness of less than 20 nm.

10. Membrane according to claim 1, at least one portion of the area of at least one of the opposite large faces of the membrane being free of any covering coating it.

11. Process for manufacturing a membrane according to claim 1, the process involving:
the provision of an n-doped silicon substrate which has an electrical resistivity of between 1 Ω·cm and 10 Ω·cm, and of an electrochemical cell including an electric generator, a cathode and an anode electrically powered by the electric generator,
the electrochemical cell containing an aqueous electrolyte including hydrofluoric acid in a mass concentration of greater than or equal to 10%,
the substrate being arranged between the anode and the cathode,
at least one of the faces of the silicon substrate being in contact with the electrolyte,
the cathode and the face of the silicon substrate opposite the cathode, known as the “front face”, being in contact with the electrolyte, the front face of the silicon substrate being distant from the cathode,
electrochemical etching of the substrate by applying with the electric generator a constant electric voltage of greater than or equal to 10 V between the anode and the cathode, so as to form blind pores in the substrate, each of which has a diameter of less than or equal to 400 nm and which extend from one face of the substrate to a depth of greater than or equal to 10 μm, the rear face of the substrate being illuminated during the electrochemical etching with light radiation so as to generate charge carriers in the substrate, and
ablation of part of the substrate so that said pores are open and pass through at least a portion of the substrate entirely across its thickness.

12. Process according to claim 11, the aqueous electrolyte having a mass concentration of hydrofluoric acid of greater than or equal to 15%.

13. Process according to claim 12, the aqueous electrolyte having a mass concentration of hydrofluoric acid of greater than or equal to 30%.

14. Process according to claim 13, the aqueous electrolyte having a mass concentration of hydrofluoric acid of greater than or equal to 35%.

15. Process according to claim 11, involving, prior to electrochemical etching, illumination of the substrate with light radiation so as to generate charge carriers in the substrate.

16. Process according to claim 11, the electrochemical etching being performed until the pores each extend from one face of the support to a depth greater than or equal to 30 μm.

17. Process according to claim 11, the electrochemical etching being performed at a constant electrical voltage of between 25 V and 100 V.

18. Process according to claim 11, the substrate having a thickness, prior to electrochemical etching, of between 100 μm and 3 mm.

19. Process according to claim 11, the ablation being performed by mechanical polishing, wet chemical etching and/or dry deep etching.

20. Process according to claim 11, including subsequent treatment of the membrane to render the membrane hydrophilic or hydrophobic.

21. Method for characterizing a colony of microorganisms, involving:
culturing the microorganisms in a nutrient medium, the microorganisms being arranged on one face of a membrane according to claim 1, the membrane being in contact with the nutrient medium in such a way that the nutrient medium is transferred via the pores to the microorganisms,
illuminating the microorganisms in transmission through the membrane by light radiation having at least one wavelength of between 5 μm and 25 μm and acquiring the light radiation which has interacted with the microorganisms.
